(19) United States
(12) Patent Application Publication
(10) Pub. No.: US 2025/0090219 A1
(43) Pub. Date: Mar. 20, 2025

(54) ENDOSCOPIC TREATMENT TOOL

(71) Applicant: OLYMPUS MEDICAL SYSTEMS CORP., Tokyo (JP)

(72) Inventor: Kirara SHIIBA, Akiruno-shi (JP)

(21) Appl. No.: 18/890,165

(22) Filed: Sep. 19, 2024

Related U.S. Application Data
(60) Provisional application No. 63/583,983, filed on Sep. 20, 2023.

(51) Int. Cl. A61B 18/14; A61B 18/00; A61B 90/00

(52) U.S. Cl. CPC A61B 18/1445; A61B 90/03; A61B 2018/00172; A61B 2018/00196; A61B 2018/00202; A61B 2090/034

(57) ABSTRACT

An endoscopic treatment tool can include a sheath and a wire inserted through the sheath. The wire can be movable forward and backward in a longitudinal direction of the sheath. The endoscopic treatment tool can further include a forceps, the forceps including a first forceps piece and a second forceps piece. The forceps can be supported or configured to be openable and closable. A connecting body can be connected to a distal end of the wire and can open and closes the forceps. When the forceps are in an open state, the first forceps piece and the second forceps piece include abutment surfaces abutting a distal end portion of the connecting body in the longitudinal direction.

### TECHNICAL FIELD

The present disclosure relates to an endoscope treatment tool. Priority is claimed on U.S. Provisional Application No. 63/583,983, filed Sep. 20, 2023, the content of which is incorporated herein by reference.

### BACKGROUND

In endoscopic treatment, endoscopic treatment tools such as hemostatic forceps can be used to cauterize a bleeding treatment target to stop the bleeding. Hemostatic forceps pass a high-frequency current through a treatment target while holding the target, thereby cauterizing the treatment target to stop bleeding.

For example, endoscopic forceps described in Patent Document 1 include an insertion tube inserted into a living body, a support member connected to a distal end of the insertion tube, a pivot pin provided in the support member, a forceps member pivotably supported on the pivot pin, a cam groove formed in the forceps member, an operation unit connected to a proximal end of the insertion tube, a forward and backward moving member that moves forward and backward in the insertion tube in an axis direction thereof in accordance with an operation of the operation unit, an insertion hole formed in the forward and backward moving member, and a cam pin that is inserted into the insertion hole and engages with the cam groove. When the cam pin moves along the cam groove with forward and backward movements of the forward and backward moving member, the forceps member rotates around the pivot pin due to the engagement of the cam pin with the cam groove to perform opening and closing movements.

### PATENT DOCUMENTS

[Patent Document 1] Japanese Unexamined Patent Application, First Publication No. 2012-045031

### SUMMARY

In a forceps opening and closing mechanism using a cam groove (slide groove) like that of the endoscopic forceps described in Patent Document 1, when a forward and backward moving member (a connecting body) that moves forward and backward in an axial direction of an insertion tube is eccentric with respect to the axial direction, there is a risk of an opening and closing axis of the forceps deviating from a central axis of the insertion tube, resulting in one-sided opening.

In light of the above circumstances, the presently disclosed endoscopic treatment tool can help to inhibit or reduce the occurrence of one-sided opening.

An endoscopic treatment tool of the present disclosure can a sheath, a wire inserted through the sheath and movable forward and backward in a longitudinal direction of the sheath, forceps that can include a first forceps piece and a second forceps piece and can be supported to be openable and closable toward a distal end side in the longitudinal direction, and a connecting body connected to a distal end of the wire and can open and close the forceps. The first forceps piece and the second forceps piece can include respective abutment surfaces that abut a distal end portion of the connecting body in the longitudinal direction when the forceps are in an open state.

It is possible to provide an endoscopic treatment tool in which occurrence of one-sided opening is inhibited.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of an overall view showing an endoscopic treatment system according to the present embodiment.

FIG. 2 illustrates an example of an overall view showing an endoscopic treatment tool of the endoscopic treatment system.

FIG. 3 illustrates an example of a cross-sectional view of a distal end portion of the endoscopic treatment tool in a closed state.

FIG. 4 illustrates an example of a cross-sectional view of the distal end portion in a half-open state.

FIG. 5 illustrates an example of a cross-sectional view of the distal end portion in an open state.

FIG. 6 illustrates an example of a plan view of the distal end portion in the open state.

FIG. 7 illustrates an example of a perspective view of a forceps piece of the distal end portion.

FIG. 8 illustrates an example of a side view showing the forceps piece.

FIG. 9 illustrates an example of a cross-sectional view of the forceps piece and a connecting member in the distal end portion in the open state.

### DETAILED DESCRIPTION OF THE DISCLOSURE

An endoscopic treatment system 300 according to one embodiment of the present disclosure will be described with reference to the drawings. Also, in each figure below, in order to make each constituent element easier to see, scales thereof may differ depending on the constituent elements.

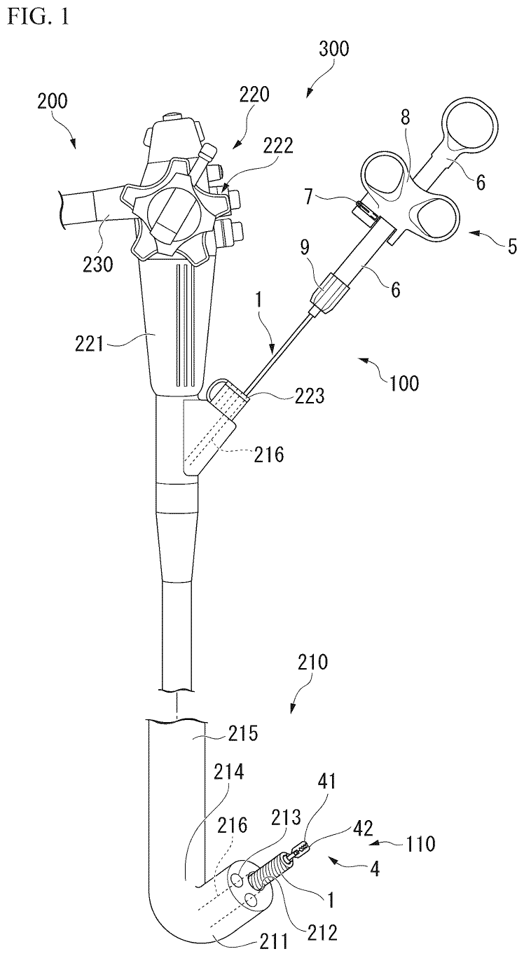

FIG. 1 illustrates an example of an overall view showing the endoscopic treatment system 300 according to the present embodiment.

### [Endoscope Treatment System 300]

As shown in FIG. 1, the endoscopic treatment system 300 can include an endoscopic treatment tool 100 and an endoscope 200. The endoscopic treatment tool (treatment tool) 100 can be inserted into the endoscope 200 for use.

### [Endoscope 200]

The endoscope 200 is a known flexible endoscope and can include an insertion unit 210 inserted into a body from its distal end, an operation unit 220 attached to a proximal end of the insertion unit 210, and a universal cord 230 attached to the operation unit 220.

The insertion unit 210 can be or include a thin elongated member that can be inserted into a lumen. The insertion unit 210 can include a distal end portion 211, a bending portion 214, and a flexible portion 215. The distal end portion 211, the bending portion 214, and the flexible portion 215 can be connected to each other in order from the distal end side. A channel 216 for inserting the treatment tool 100 can be provided in the insertion unit 210. A distal end opening portion 212 of the channel 216 and an imaging unit 213 can be provided in the distal end portion 211.

The imaging unit 213 can include an imaging element such as a CCD or CMOS and can image a portion serving as a treatment target. The bending portion 214 can bend in response to a user's operation of the operation unit 220. The flexible portion 215 can be or include a flexible tubular portion.

The operation unit 220 can be connected to the flexible portion 215. The operation unit 220 can include a grip 221, an input unit 222, and a forceps port 223. The grip 221 can be a member supported by the user. The input unit 222 can receive an operation input for bending the bending portion 214. The forceps port 223 can be a proximal end opening portion of the channel 216.

The universal cord 230 can connect the endoscope 200 to an external device. An imaging cable or optical fiber cable that outputs image signals captured by the imaging unit 213 to the outside can be inserted into the universal cord 230.

### [Endoscope Treatment Tool 100]

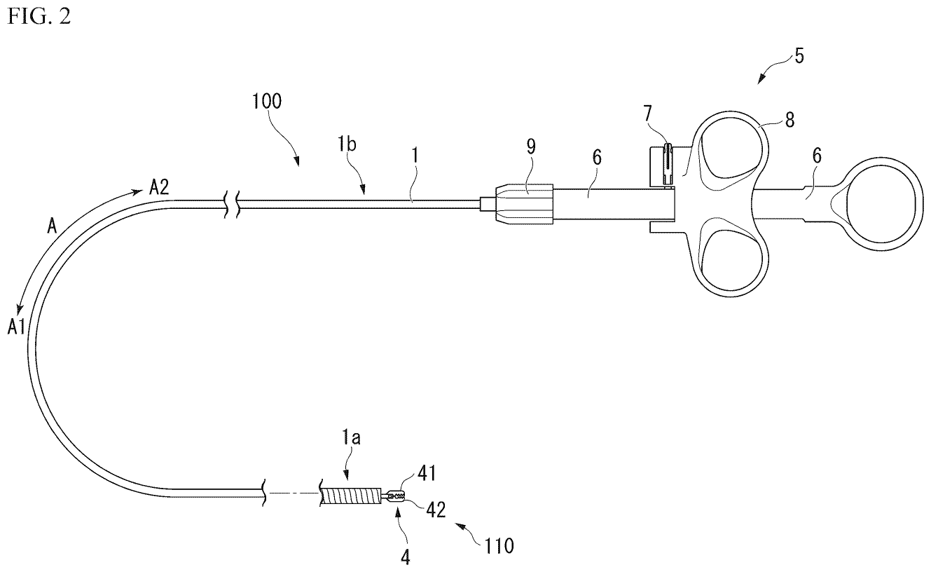

FIG. 2 illustrates an example of an overall view showing the endoscopic treatment tool 100.

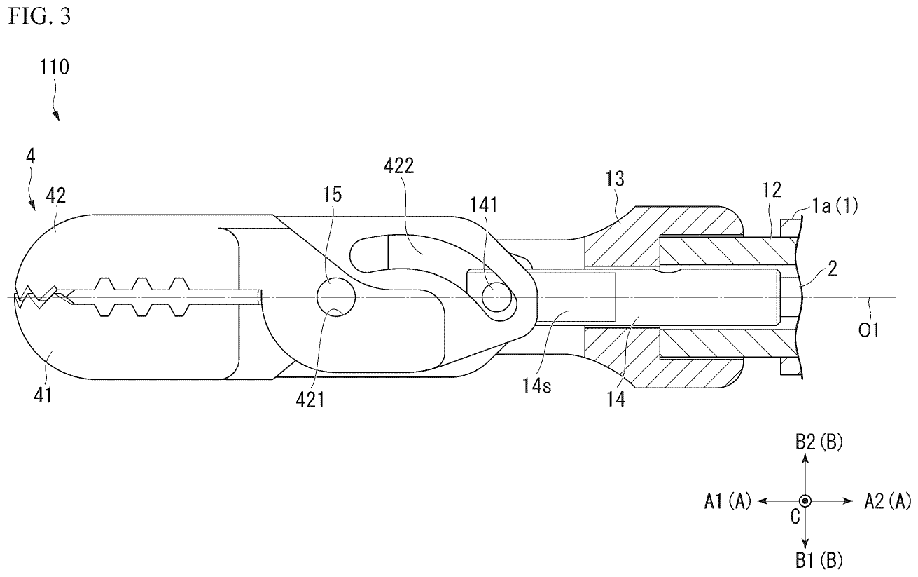

The endoscopic treatment tool (treatment tool) 100 can include a sheath 1, an operation wire (wire) 2 (see FIG. 3), forceps (jaws) 4, a wire operation unit 5, a connection member 12, a support member (cover member, support body) 13, and a connecting member (connecting body) 14. In the following description, as shown in FIG. 2, in a longitudinal direction A along a central axis of the treatment tool 100, a side inserted into a patient's body will be referred to as a “distal end side (distal side) A1” and the wire operation unit 5 side will be referred to as a “proximal end side (proximal side) A2.”

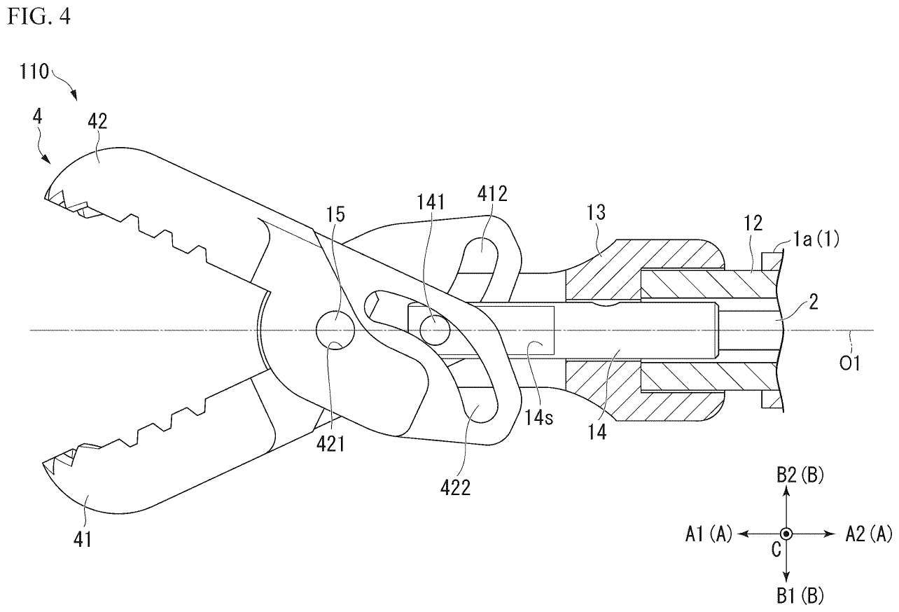

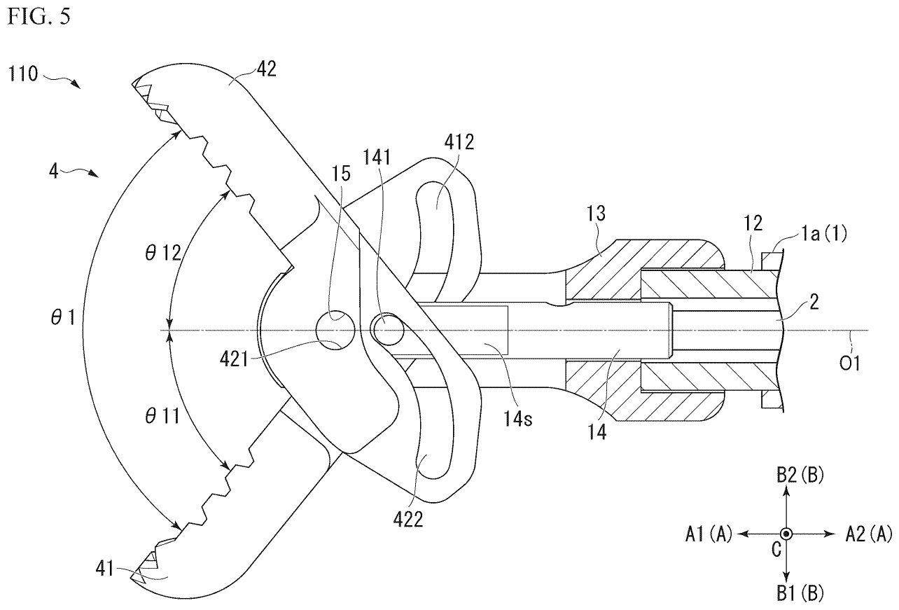

FIG. 3 illustrates an example of a cross-sectional view of a distal end portion of the treatment tool 100 in a closed state. FIG. 4 illustrates an example of a cross-sectional view of the distal end portion of the treatment tool 100 in a half-open state. FIG. 5 illustrates an example of a cross-sectional view of the distal end portion of the treatment tool 100 in an open state. The forceps 4, the support member 13, and the connecting member 14 provided in the distal end portion of the treatment tool 100 can form a “treatment unit 110” that treats an affected site.

In the following description, as shown in FIGS. 3, 4, and 5, directions in which the forceps 4 open and close will be referred to as “opening and closing directions B” or “up and down directions B.” A direction in which a forceps piece (first forceps piece) 41 opens will be referred to as “downward B1” in the opening and closing directions B, and a direction in which a forceps piece (second forceps piece) 42 opens will be referred to as “upward B2” in the opening and closing directions B.

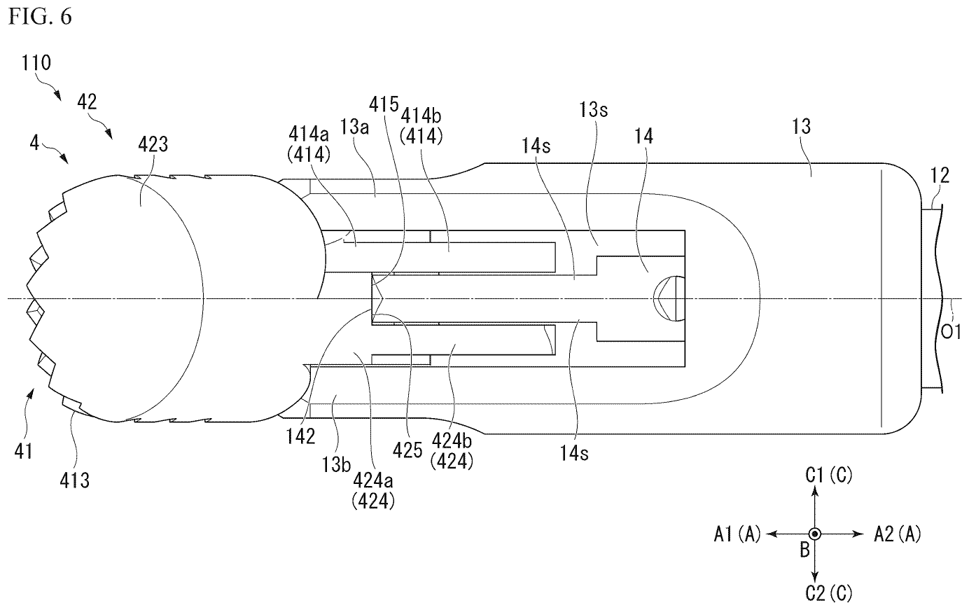

FIG. 6 illustrates an example of a plan view showing the distal end portion of treatment tool 100 in the open state.

As shown in FIG. 6, a direction perpendicular to the longitudinal direction A and the opening and closing directions B will be referred to as a “width direction C” or “left and right directions C.” A direction facing left when viewed from the distal end side A1 toward the proximal end side A2 will be referred to as a “left side C1” in the width direction C, and a direction facing right will be referred to as a “right side C2” in the width direction C.

The sheath 1 can be an elongated resin member that can be flexible and insulating and can extends from a distal end 1a to a proximal end 1b thereof as shown in FIG. 2. The sheath 1 can include an outer diameter that allows it to be inserted into the channel 216 of the endoscope 200 and can move forward and backward through the channel 216. With the sheath 1 inserted into the channel 216, the distal end 1a of the sheath 1 can protrude and retract from the distal end opening portion 212 of the channel 216. In addition, a cylindrically wound metal coil sheath can be provided inside the sheath 1.

The operation wire 2 can be inserted through an internal space of the sheath 1 and the coil sheath described above. A distal end of the operation wire 2 can be connected to the forceps 4 via the connecting member 14, and a proximal end of the operation wire 2 can be connected to the wire operation unit 5.

The wire operation unit 5 can be provided on the proximal end side A2 of the sheath 1. The wire operation unit 5 can include a handle body 6, a connector 7, a slider 8, and a rotation handle 9. In the present embodiment, the connector 7 can be attached to the slider 8.

The connector 7 can be connected to a high-frequency power supply device and can be electrically and physically connected to the proximal end of the operation wire 2. The connector 7 can be connected to the high-frequency power supply device, for example, via an A-chord (an active chord). The connector 7 can supply a high-frequency current supplied from the high-frequency power supply device to the forceps 4 via the operation wire 2.

The slider 8 can be attached to be movable forward and backward along a slit formed in the handle body 6 and extending in the longitudinal direction A. The slider 8 can be moved forward and backward in the longitudinal direction A relative to the handle body 6.

A proximal end portion of the operation wire 2 can be connected to the slider 8. The operation wire 2 can passe through the internal space of the sheath 1 and an internal space of the handle body 6 and extends to the slider 8. The operation wire 2 can move forward and backward when the surgeon moves the slider 8 forward and backward relative to the handle body 6.

The rotation handle 9 can be provided on the handle body 6 on the distal end side A1 of the slider 8. The rotation handle 9 can be rotatable around the longitudinal direction A relative to the handle body 6. In addition, the rotation handle 9 can be connected to the operation wire 2 that can be inserted through an internal space of the rotation handle 9. For that reason, when the rotation handle 9 is rotated around the longitudinal direction A relative to the handle body 6, the operation wire 2 can rotate around the longitudinal direction A together with the rotation handle 9.

A surgeon can move the operation wire 2 forward and backward in the longitudinal direction A by moving the slider 8 forward and backward in the longitudinal direction A. Further, the surgeon can rotate the operation wire 2 around the longitudinal direction A by rotating the rotation handle 9 around the longitudinal direction A relative to the handle body 6.

Next, the distal end portion of the treatment tool 100 will be described. Central axes of the sheath 1 and the operation wire 2 extending in the longitudinal direction A can coincide with each other in the distal end portion of the treatment tool 100, and as shown in FIGS. 3, 4, and 5, the central axis of the sheath 1 and the operation wire 2 will be referred to as a central axis O1.

The connection member 12 can be or include a cylindrical member that extends with the central axis O1 as a center, and its proximal end can be connected to a distal end of the coil sheath. In addition, the operation wire 2 can be inserted into an internal space of the connection member 12. The distal end of the sheath 1 is located slightly on the distal end side A1 from the distal end of the coil sheath and can be connected to an outer circumferential surface of the connection member 12. The connection member 12 can be made of an insulating material.

The support member 13 can be or include a cylindrical member that extends with the central axis O1 as a center. The support member 13 can include a first support piece 13a and a second support piece 13b that can extend to the distal end side A1 as shown in FIG. 6.

The first support piece 13a and the second support piece 13b can be provided in the width direction C on either side of the central axis O1. In an example, the first support piece 13a can be provided on the left side C1 from the central axis O1 and the second support piece 13b can be provided on the right side C2 from the central axis O1. A support space 13s can be formed between the first support piece 13a and the second support piece 13b in the width direction C.

In addition, as shown in FIGS. 3, 4, and 5, a proximal end of the support member 13 can be provided with a concave shape that opens to the proximal end side A2. A distal end of the connection member 12 can be disposed to be surrounded by an inner circumferential surface of the concave shape.

Further, the support member 13 can be supported by the connection member 12 to be rotatable around the longitudinal direction A relative to the connection member 12. The connecting member 14 can be connected to the distal end of the operation wire 2 and can be inserted into an internal space of the support member 13 and the support space 13s.

The connecting member 14 can be or include a cylindrical member that extends with the central axis O1 as a center, and as shown in FIG. 6, flat surfaces (planar portions) 14s can be provided on the left side C1 and the right side C2. In addition, the planar portions 14s can be provided with protrusion portions (protrusions, slide pins) 141 that protrude on the left side C1 and the right side C2. The protrusion portions 141 can be provided integrally with the connecting member 14, or can be pins or the like that are separate parts from the connecting member 14.

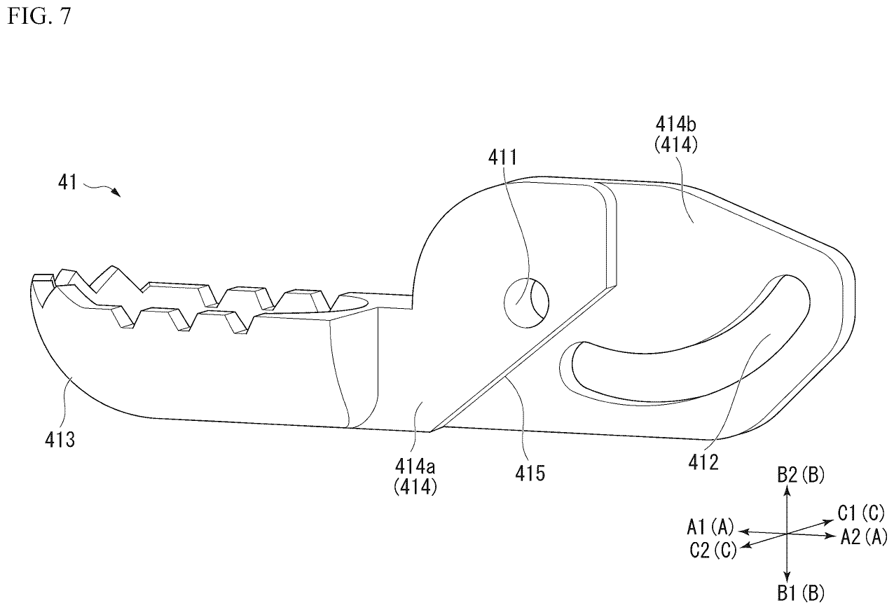

The forceps 4 can include a first forceps piece 41 and a second forceps piece 42. FIG. 7 illustrates an example of a perspective view showing the forceps piece (first forceps piece) 41.

The first forceps piece 41 can include a first forceps cup (forceps cup) 413 and a first plate portion (plate portion) 414. The first forceps cup 413 can be formed in a hemispherical shape and opens toward the second forceps piece 42 side (upward B2) in the opening and closing directions B. An opening edge of the first forceps cup 413 can have an uneven shape in the opening and closing directions B when the forceps 4 are in a closed state.

The first plate portion 414 can be provided on the proximal end side A2 of the first forceps cup 413, and is formed in a plate shape. The first plate portion 414 can include a first thick plate portion (thick plate portion, distal section) 414a connected to a proximal end of the first forceps cup 413, and a first thin plate portion (thin plate portion, proximal section) 414b that has a plate thickness smaller in the width direction C than that of the first thick plate portion 414a.

As shown in FIG. 7, the first thick plate portion 414a can be provided with a shaft hole 411 penetrating in the width direction C. Also, the first thin plate portion 414b can be provided with a slide groove 412 penetrating in the width direction C. As shown in FIG. 7, the slide groove 412 can be a groove having a arc shape. In addition, a first abutment portion (abutment portion, abutment surface) 415, which can be or include a flat surface (second flat surface) extending in the width direction C, can be provided at a boundary portion (step portion, step protrusion) between the first thick plate portion 414a and the first thin plate portion 414b.

The second forceps piece 42 can have or be formed in the same shape as the first forceps piece 41, and can be disposed to face a side opposite to the first forceps piece 41 in the opening and closing directions B.

As shown in FIG. 6, a second forceps cup (forceps cup) 423 can be provided at a distal end of the second forceps piece 42, and can be formed in a hemispherical shape and can open toward the first forceps piece 41 side (downward B1) in the opening and closing directions B. An opening edge of the second forceps cup 423 can be formed with an uneven shape in the opening and closing directions B when the forceps 4 are in the closed state.

The uneven shapes formed on the opening edges of the first forceps cup 413 and the second forceps cup 423 can have shapes that engage with each other when the forceps 4 are in the closed state, as shown in FIG. 3.

Also, as shown in FIG. 6, a second plate portion (plate portion) 424 which can be formed in a plate shape can be provided at a proximal end of the second forceps cup 423 of the second forceps piece 42. Similarly to the first plate portion 414, the second plate portion 424 can include a second thick plate portion (thick plate portion, distal section) 424a connected to the proximal end of the second forceps cup 423, and a second thin plate portion (thin plate portion, proximal section) 424b having a plate thickness in the width direction C thinner than that of the second thick plate portion 424a.

As shown in FIGS. 3, 4, and 5, the second thick plate portion 424a can be provided with a shaft hole 421 penetrating in the width direction C. Also, the second thin plate portion 424b can be provided with a slide groove 422 penetrating in the width direction C. The slide groove 422 can be a groove having a arc shape. In addition, a second abutment portion (abutment portion, abutment surface) 425, which can be or include a flat surface (second flat surface) extending in the width direction C, can be provided at a boundary portion (step portion, step protrusion) between the second thick plate portion 424a and the second thin plate portion 424b.

As shown in FIG. 6, the first forceps piece 41 and the second forceps piece 42 can be disposed in the width direction C with the connecting member 14 interposed between the first thin plate portion 414b and the second thin plate portion 424b. Also, the first thick plate portion 414a and the second thick plate portion 424a can be disposed adjacent to each other in the width direction C.

In an example, through holes penetrating in the width direction C can be formed in the first support piece 13a and the second support piece 13b of the support member 13. A shaft pin (shaft member) 15 can engage with and can be attached to the above-mentioned through holes of the first support piece 13a and the second support piece 13b. In addition, as shown in FIGS. 3, 4, and 5, the shaft pin 15 can engage with and can be attached to the shaft hole 411 of the first forceps piece 41 and the shaft hole 421 of the second forceps piece 42.

The shaft pin 15 may be a cylindrical rotating shaft member having a central axis extending in the width direction C. The first forceps piece 41 and the second forceps piece 42 can be rotatably supported by the first support piece 13a and the second support piece 13b of the support member 13 via the shaft pin 15. The first forceps piece 41 and the second forceps piece 42 can rotate around the central axis (rotation axis) of the shaft pin 15.

Further, the protrusion portions (protrusions, slide pins) 141 can be provided on the left side C1 and the right side C2 of the connecting member 14 and can be inserted respectively into the slide grooves 412 and 422 of the first forceps piece 41 and the second forceps piece 42.

As shown in FIG. 3, when the forceps 4 are in the closed state, the protrusion portions 141 can be located near proximal ends of the slide grooves 412 and 422. When the forceps 4 is in the closed state, when the connecting member 14 moves toward the distal end side A1, the protrusion portions 141 can move near middle portions of the slide grooves 412 and 422 in the longitudinal direction A, as shown in FIG. 4. Also, when the protrusion portions 141 move toward the distal end side A1 along the slide grooves 412 and 422, the first forceps piece 41 and the second forceps piece 42 can rotate around the central axis of the shaft pin 15 serving as a rotation center, and open in the opening and closing directions B, as shown in FIG. 4.

When the forceps 4 are in the half-open state as shown in FIG. 4, when the connecting member 14 moves further toward the distal end side A1, the protrusion portions 141 can move near distal end sides of the slide grooves 412 and 422, as shown in FIG. 5. In this case, the first forceps piece 41 and the second forceps piece 42 can further rotate around the central axis of the shaft pin 15 serving as the rotation center, and are in the open state shown in FIG. 5.

The surgeon can move the operation wire 2 forward and backward in the longitudinal direction A by operating the wire operation unit 5. As the operation wire 2 moves forward and backward, the connecting member 14 connected to the distal end of the operation wire 2 can move forward and backward in the longitudinal direction A together with the operation wire 2. In addition, by moving the connecting member 14 forward and backward to move positions of the protrusion portions 141 in the longitudinal direction A, the first forceps piece 41 and the second forceps piece 42 can be rotated and the forceps 4 can be opened and closed. That is, the surgeon can open and close the forceps 4 by operating the wire operation unit 5 to move the operation wire 2 forward and backward.

In addition, the operation wire 2 and the forceps 4 can be connected to each other via the connecting member 14. The support member 13 supporting the forceps 4 can be supported rotatably around the longitudinal direction A relative to the connection member 12 connected to the distal end of the sheath 1. For that reason, the surgeon can rotate the operation wire 2 and the forceps 4 by rotating the rotation handle 9 around the longitudinal direction A relative to the handle body 6.

Here, a procedure using the endoscopic treatment system 300 of the present embodiment (a method for using the endoscopic treatment system 300) will be described. Specifically, an incision or dissection treatment and a hemostatic treatment of a lesioned site in an endoscopic treatment such as endoscopic submucosal dissection (ESD) will be described.

An incision or dissection treatment often accompanies bleeding. When bleeding occurs, the surgeon performs a hemostatic treatment. A hemostatic treatment is a treatment to stop bleeding by cauterizing an ulcerated site after a lesioned site has been dissected or a bleeding site that has bled during an incision or dissection treatment.

In order to dispose the forceps 4 at an appropriate treatment position, the surgeon can rotate the rotation handle 9 to rotate the operation wire 2 and the forceps 4. Even when the rotation handle 9 rotates, the connector 7 attached to the slider 8 does not rotate, and thus the A-chord (active chord) connected to the connector 7 does not get tangled in the wire operation unit 5.

The surgeon can apply high-frequency current to the operation wire 2 via the connector 7. The high-frequency current applied to the operation wire 2 can be applied to the forceps 4 via the connecting member 14. The surgeon can hold the bleeding site with the forceps 4 to which the high-frequency current is applied, and can cauterize the bleeding site to stop the bleeding.

The surgeon continues the above-mentioned operation (treatment) if necessary, and finally can resect the lesioned site and completes the ESD procedure.

Next, a structure for preventing one-sided opening in the treatment unit 110 will be described.

As shown in FIG. 5, an angle at which the forceps 4 are open in the open state is defined as an opening angle θ1. The opening angle θ1 of the forceps 4 can be defined as the sum of a first opening angle θ11, which is an angle at which the first forceps piece 41 opens, and a second opening angle θ12, which is an angle at which the second forceps piece 42 opens, in the forceps 4 in the open state.

Here, the first opening angle θ11 can be an angle formed between the central axis O1 and the first forceps piece 41 in the forceps 4 in the open state. The second opening angle θ12 can be an angle formed between the central axis O1 and the second forceps piece 42 in the forceps 4 in the open state. The opening angle θ1 of the forceps 4 can be an angle formed between the first forceps piece 41 and the second forceps piece 42.

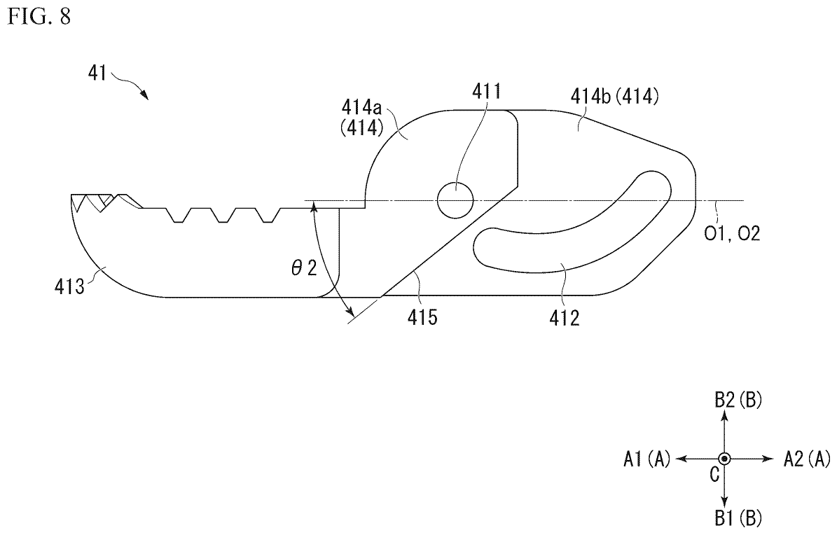

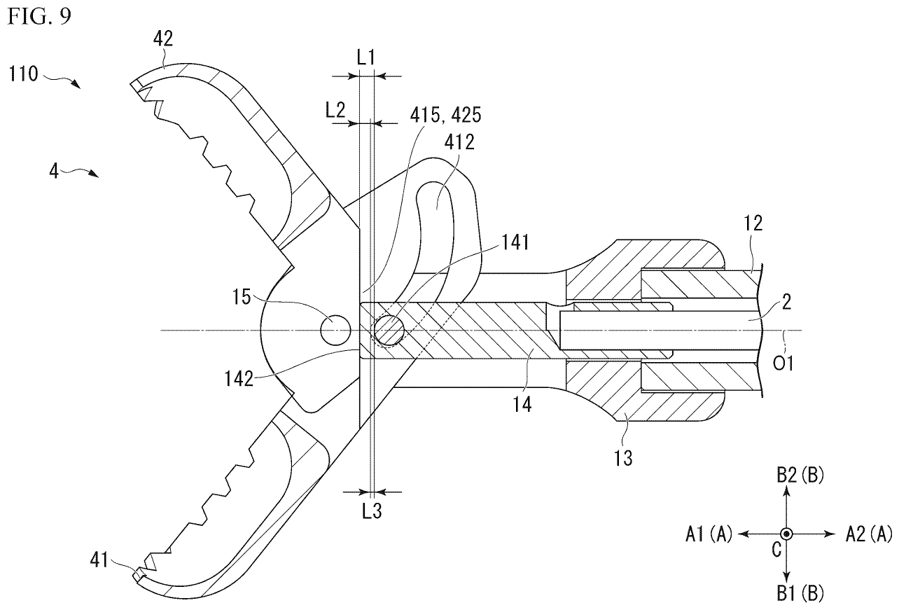

FIG. 8 illustrates an example of a side view showing the forceps piece (first forceps piece) 41. FIG. 9 illustrates an example of a cross-sectional view showing the forceps pieces 41 and 42 and the connecting member 14 in the forceps 4 in the open state.

As shown in FIG. 8, the direction in which the first forceps piece 41 (and the second forceps piece 42) in the forceps 4 in the closed state extends can be defined as a central axis (axial direction) O2. The central axis O2 is a central axis of the forceps 4 in the closed state and can be an axis passing through a rotation center of the forceps 4. That is, the central axis O2 can be an axis passing through the central axis (rotation axis) of the shaft pin 15. In addition, in the present embodiment, the central axis O2 of the forceps 4 can coincide with the central axis O1 of the treatment tool 100.

Further, as shown in FIG. 8, the angle formed between the central axis O2 of the first forceps piece 41 and the first abutment portion (abutment portion) 415 can be defined as an abutment portion angle θ2 (abutment surface angle). The angle between the central axis O2 of the first forceps piece 41 and the first abutment portion 415 can be equal to the angle between the central axis O2 of the second forceps piece 42 and the second abutment portion 425. Equal can include a design error range of 1 mm. For that reason, the angle between the central axis O2 of the forceps pieces 41 and 42 and the abutment portions 415 and 425 can be referred to as the abutment portion angle θ2.

As shown in FIG. 9, in the treatment unit 110 in the open state, a distal end portion 142 of the connecting member 14 and the abutment portions 415 and 425 can face and abut each other in the longitudinal direction A. For that reason, in the treatment unit 110 in the open state, a posture of the connecting member 14 can be regulated by the abutment portions 415 and 425, and a state in which the central axis of the connecting member 14 and the central axis O2 of the forceps 4 approximately coincide with each other can be maintained.

For example, in the treatment unit in the open state, when the distal end portion of the connecting member and the abutment portions of the forceps do not abut, the connecting member can take a posture in which the central axis of the connecting member can be offset from the central axis of the forceps. In that case, the opening angle of the first forceps piece (first opening angle) and the opening angle of the second forceps piece (second opening angle) can differ, and one-sided opening may occur.

In the treatment unit 110 in the open state, by abutting the distal end portion 142 of the connecting member 14 with the abutment portions 415 and 425, it is possible to inhibit deviation of the central axis of the connecting member 14 from the central axis O2 of the forceps 4, thereby inhibiting occurrence of one-sided opening. By inhibiting one-sided opening of the forceps 4, operability of the endoscopic treatment tool 100 can be improved when the hemostatic treatment is performed.

Also, when the forceps 4 are in the open state, flat surfaces (first flat surface) of the distal end portion 142 of the connecting member 14 and the abutment portions 415 and 425 can abut each other in the longitudinal direction A.

A position of the first forceps piece 41 open at the first opening angle θ11 can be referred to as a first forceps fixing position (forceps fixing position). Also, a position of the second forceps piece 42 can open at the second opening angle θ12 can be referred to as a second forceps fixing position (forceps fixing position).

When the distal end portion 142 of the connecting member 14 abuts the abutment portions 415 and 425, the first forceps piece 41 can be fixed at the first forceps fixing position, and the second forceps piece 42 can be fixed at the second forceps fixing position. The first forceps fixing position and the second forceps fixing position can be predetermined positions that are set in advance. In addition, the first opening angle θ11 and the second opening angle θ12 can be predetermined angles that are can be in advance. That is, the opening angle θ1 of the forceps 4 can be a predetermined angle that is set in advance.

By abutting the distal end portion 142 of the connecting member 14 with the abutment portions 415 and 425, the surgeon can easily fix the first forceps piece 41 and the second forceps piece 42 in predetermined positions (forceps fixing positions) when the forceps 4 are open. Also, the first opening angle θ11 and the second opening angle θ12 can be equal angles, but they may be different angles. Equal can include a design error range of 1 mm.

As shown in FIG. 9, when the treatment unit 110 is in the open state, the abutment portions 415 and 425 of the forceps 4 can be located between the shaft pin 15 serving as the rotation axis of the forceps 4 and the slide grooves 412 and 422 of the forceps 4 in the longitudinal direction A.

A dimension L1 shown in FIG. 9 can be a distance (dimension) in the longitudinal direction A between the abutment portions 415 and 425 of the forceps 4 and the protrusion portions 141 of the connecting member 14 in the open state of the treatment unit 110. A dimension L2 can be a distance (dimension) in the longitudinal direction A between the abutment portions 415 and 425 of the forceps 4 and distal ends of the slide grooves 412 and 422 in the open state of the treatment unit 110. Also, a dimension L3 can be a distance (dimension) in the longitudinal direction A between the protrusion portions 141 of the connecting member 14 and the distal ends of the slide grooves 412 and 422 in the open state of the treatment unit 110.

Here, gaps can be maintained between the protrusion portions 141 of the connecting member 14 and the distal ends of the slide grooves 412 and 422 in the open state of the treatment unit 110. That is, the dimension L3 can be greater than 0 mm, and the protrusion portions 141 of the connecting member 14 do not abut the distal ends of the slide grooves 412 and 422.

Also, the dimension L1 (the distance from the abutment portions 415 and 425 to the protrusion portions 141) can be greater than the dimension L2 (the distance from the abutment portions 415 and 425 to the distal ends of the slide grooves 412 and 422).

By setting the dimensions L1, L2, and L3 as described above, in the treatment unit 110 in the open state, the movement of the protrusion portions 141 toward the distal end side A1 is not hindered by the distal ends of the slide grooves 412 and 422, and the distal end portion 142 of the connecting member 14 can reliably abut the abutment portions 415 and 425.

Also, when the treatment unit 110 opens, the abutment portion 415 of the first forceps piece 41 and the abutment portion 425 of the second forceps piece 42 can simultaneously abut the distal end portion 142 of the connecting member 14.

In addition, the above-mentioned opening angle θ1 and the abutment portion angle θ2 can have a relationship of θ2=90°−θ½. By doing so, when the forceps 4 are open and the distal end portion 142 of the connecting member 14 abuts the abutment portions 415 and 425, the first opening angle θ11 and the second opening angle θ12 can become equal angles, and the first forceps piece 41 and the second forceps piece 42 can be open evenly. Equal can include a design error range of 1 mm.

The endoscopic treatment tool 100 according to the present embodiment can include the sheath 1, the operation wire (wire) 2 that is inserted through the sheath 1 and movable forward and backward in the longitudinal direction A of the sheath, the forceps 4 that include the first forceps piece 41 and the second forceps piece 42 and can be supported to be openable and closable toward the distal end side A1 in the longitudinal direction A, and the connecting member 14 connected to the distal end of the operation wire 2 and can open and close the forceps 4. In addition, the first forceps piece 41 and the second forceps piece 42 can include the abutment portions 415 and 425 that abut the distal end portion 142 of the connecting member 14 in the longitudinal direction A when the forceps 4 are in the open state.

As a result, it is possible to inhibit deviation of the central axis of the connecting member 14 from the central axis O2 of the forceps 4, thereby inhibiting occurrence of one-sided opening.

Although one embodiment of the present disclosure has been described above in detail with reference to the drawings, specific configurations thereof are not limited to the present embodiment, and design modifications within the scope of the present disclosure are also included. In addition, the constituent elements shown in the above-mentioned embodiment and modified examples can be appropriately combined to constitute the present disclosure.

### Modified Example 1

In the above embodiment, the treatment unit 110 is hemostatic forceps that cauterize an affected site to stop bleeding, but a type of treatment unit is not limited thereto. The treatment unit may be biopsy forceps that collect biological tissue.

### Modified Example 2

In the above embodiment, the distal end portion 142 of the connecting member 14 and the abutment portions 415 and 425 of the forceps 4 have flat surfaces, and the flat surfaces abut each other when the treatment unit 110 is in the open state, but the distal end portion of the connecting member and the abutment portions of the forceps are not limited thereto.

The distal end portion of the connecting member and the abutment portions of the forceps can have or be formed in any shapes that abut each other when the treatment unit is in the open state and that can inhibit one-sided opening of the forceps, and the distal end portion of the connecting member and the abutment portions of the forceps can have uneven shapes, for example. In addition, either the distal end portion of the connecting member or the abutment portions of the forceps can be flat, and the other can be a surface having an uneven shape. The embodiments described and illustrated herein are examples only and are not to be considered as limiting. Additions, omissions, substitutions, and other modifications can be made without departing from the spirit or scope of the present disclosure. Accordingly, the present disclosure is not to be considered as being limited by the foregoing description and is only limited by the scope of the appended claims.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. An endoscopic treatment tool comprising:
a sheath;
a wire inserted through the sheath and movable forward and backward in a longitudinal direction of the sheath;
forceps including a first forceps piece and a second forceps piece, the forceps supported to be openable and closable; and
a connecting body connecting a distal end of the wire and the forceps, wherein, when the forceps are in an open state, the first forceps piece and the second forceps piece include abutment surfaces abutting a distal end portion of the connecting body in the longitudinal direction.

2. The endoscope treatment tool according to claim 1, wherein the first forceps piece is fixed at a first forceps fixing position open at a predetermined opening angle when the distal end portion of the connecting body abuts the abutment surface of the first forceps piece.

3. The endoscope treatment tool according to claim 2, wherein the second forceps piece is fixed at a second forceps fixing position open at a predetermined opening angle when the distal end portion of the connecting body abuts the abutment surface of the second forceps piece.

4. The endoscope treatment tool according to claim 1, wherein the distal end portion of the connecting body includes a first flat surface abutting the abutment surfaces when the forceps are in the open state, and wherein the abutment surfaces each include a second flat surface abutting the distal end portion of the connecting body when the forceps are in the open state.

5. The endoscope treatment tool according to claim 1, wherein a central axis of the connecting body and a central axis of the forceps coincide with each other when the forceps are in the open state.

6. The endoscope treatment tool according to claim 1, wherein the connecting body includes protrusions protruding in a width direction, wherein the first forceps piece and the second forceps piece include slide grooves into which the protrusions are inserted, and wherein the forceps are opened and closed by the protrusions moving along the slide grooves.

7. The endoscope treatment tool according to claim 6, wherein the abutment surfaces are located between a rotation axis of the forceps and the slide grooves in the longitudinal direction when the forceps are in the open state.

8. The endoscope treatment tool according to claim 6, wherein the protrusions and distal ends of the slide grooves are disposed not to abut each other in the longitudinal direction when the forceps are in the open state.

9. The endoscope treatment tool according to claim 6, wherein the protrusions move toward a proximal end side in the longitudinal direction when the forceps are brought into a closed state from the open state.

10. The endoscope treatment tool according to claim 1, wherein a first abutment surface and a second abutment surface simultaneously abut the distal end portion of the connecting body when the forceps are brought into the open state from a closed state, wherein the first abutment surface is the abutment surface of the first forceps piece, the second abutment surface is the abutment portion of the second forceps piece.

11. The endoscope treatment tool according to claim 1, wherein an opening angle of the first forceps piece relative to a central axis of the forceps and an opening angle of the second forceps piece relative to the central axis of the forceps are equal when the forceps are in the open state.

12. The endoscope treatment tool according to claim 1, wherein, when an opening angle of the forceps in the open state is defined as θ1, and an abutment surface angle, which is an angle between the abutment surfaces and a central axis of the forceps when the forceps are in a closed state, is defined as θ2, the opening angle and the abutment surface angle satisfy a relationship of θ2=90°−θ½.

13. The endoscopic treatment tool according to claim 1 further comprising:
a wire operation unit connected to a proximal end of the wire and configured to move the wire forward and backward.

14. The endoscopic treatment tool according to claim 13, wherein the wire operation unit includes a connector connected to the wire, and wherein the connector is configured to supply a high-frequency current to the forceps via the wire.

15. The endoscope treatment tool according to claim 4, wherein the first forceps piece and the second forceps piece each include a thick plate portion and a thin plate portion, and wherein the abutment surface is located between the thick plate portion and the thin plate portion.

16. The endoscope treatment tool according to claim 15, wherein a step portion comprises the thick plate portion, the thin plate portion and the abutment surface.

17. The endoscope treatment tool according to claim 6, wherein the protrusions are located proximally relative to the distal end of the connecting body in the longitudinal direction.

18. The endoscopic treatment tool according to claim 1, wherein the abutment surfaces are formed as step protrusions between proximal sections and distal sections of the first and second forceps pieces, and wherein the distal sections have a greater thickness than the proximal sections in a width direction perpendicular to the longitudinal direction.

19. The endoscopic treatment tool according to claim 1, wherein the connecting body is cylindrical and includes flat surfaces on opposing sides, and wherein one or more protrusions extend outward from the flat surfaces.

20. The endoscopic treatment tool according to claim 1, further comprising:
a support body rotatably supports the forceps, wherein the support body is rotatable relative to the sheath around a longitudinal axis of the sheath.
